(19) United States
(12) Patent Application Publication
(10) Pub. No.: US 2022/0256792 A1
(43) Pub. Date: Aug. 18, 2022

(54) CULTIVATION SYSTEMS FOR SEAWEEDS

(71) Applicant: W. L. Gore & Associates, Inc., Newark, DE (US)

(72) Inventor: Norman E. Clough, Landenberg, PA (US)

(21) Appl. No.: 17/622,688

(22) PCT Filed: Jun. 26, 2020
(86) PCT No.: PCT/US2020/039948

§ 371 Date: Dec. 23, 2021

Related U.S. Application Data
(60) Provisional application No. 62/867,707, filed on Jun. 27, 2019.

(51) Int. Cl. A01G 33/00; A01G 24/18; A01G 24/46; A01G 18/20; A01G 24/35; B32B 5/02; B32B 5/18

(52) U.S. Cl. CPC A01G 33/00; B32B 2266/0278; A01G 24/46; A01G 18/20; A01G 24/35; B32B 5/024; B32B 5/022; B32B 5/026; B32B 5/02; B32B 5/18; B32B 2307/72; B32B 2307/728; B32B 2307/73; B32B 2266/025; B32B 2266/0221; B32B 2266/0214; A01G 24/18

(57) ABSTRACT

Cultivation systems including a cultivation substrate configured to retain and viably maintain spores and germinated spores are disclosed. The cultivation systems may include one or more of a nutrient phase, an adhesive, a bioactive agent, a liquid containing phase. The cultivation substrates may be patterned. The cultivation systems may specifically retain and viably retain specific spore types.

### CROSS-REFERENCE TO RELATED APPLICATION

This application claims the benefit of Provisional Application No. 62/867,707, filed Jun. 27, 2019, which is incorporated herein by reference in its entirety for all purposes.

### FIELD

The present disclosure relates generally to cultivation systems, and more specifically to cultivation systems configured to retain and viably maintain spores, including seaweed spores.

### BACKGROUND

The current process to cultivate seaweed from spores involves using textured nylon “culture strings” or “seed strings” to which the spores weakly attach during a lab-based seeding process and are then nourished through external nutrient systems. The culture string containing weakly attached juvenile seaweed (gametophytes and sporophytes) is then wound onto ropes at a seaweed farm, where the ropes are subsequently placed under water. The process is inherently variable in terms of yield and throughput due in large part to the ease in which the seaweed can be damaged from, for example, currents, changes in temperature, and nutrient availability. Further, poor packaging and handling can result in damage and loss of juvenile seaweed. Current approaches to improving stability of juvenile seaweed on culture strings is focused on the surface texture of existing fibers. Indeed, fiber texture of culture strings is very important to the success of seaweed cultivation. However, improvements to surface texture are limited.

### SUMMARY

Various embodiments are directed toward cultivation systems configured to retain and viably maintain spores.

According to one example (“Example 1”), the cultivation substrate having a microstructure configured to retain and viably maintain spores, the microstructure being characterized by an average inter-fibril distance up to and including 200 μm.

According to another example (“Example 2”), the cultivation substrate having a microstructure wherein at least a portion of the cultivation system is configured to retain and viably maintain, the microstructure configured to retain spores at least partially within the microstructure of the cultivation substrate, the microstructure being characterized by an average pore size of up to and including 200 μm.

According to another example (“Example 3”) further to Example 1, the microstructure is characterized by an average inter-fibril distance from 1 to 200 μm,

According to another example (“Example 4”) further to any one of preceding Examples 1 or 2, the microstructure is characterized by an average pore size from 1 to 200 μm.

According to another example (“Example 5”) further to any one of preceding Examples 1 to 4, the cultivation system comprising a nutrient phase associated with at least a portion of the cultivation substrate.

According to another example (“Example 6”) further to Example 5, at least a portion of the nutrient phase is located within the cultivation substrate, located on the cultivation substrate, or located both within the cultivation substrate and on the cultivation substrate.

According to another example (“Example 7”) further to Example 5, the nutrient phase is present as a coating on a surface of the cultivation substrate.

According to another example (“Example 8”) further to any one of preceding Examples 5 to 7, the nutrient phase acts as a chemoattractant to selectively attract the spores to predetermined locations of the cultivation substrate to which the nutrient phase is applied or included.

According to another example (“Example 9”) further to any one of preceding Examples 5 to 8, the nutrient phase is configured to i) promote germination of and growth from the spores within the microstructure, and/or ii) maintain and/or encourage attachment to and integration within the microstructure by the spores.

According to another example (“Example 10”) further to any one of preceding Examples 1 to 9, the cultivation system comprises a liquid containing phase associated with at least a portion of the cultivation substrate.

According to another example (“Example 11”) further to preceding Example 10, at least a portion of the liquid containing phase is entrained within the microstructure, entrained on the microstructure, or entrained both within the microstructure and on the microstructure.

According to another example (“Example 12”) further to any one of preceding Examples 10 or 11, the liquid containing phase is present as a coating on a surface of the cultivation substrate.

According to another example (“Example 13”) further to any one of preceding Examples 10 to 12, the liquid containing phase comprises a hydrogel, a slurry, a paste, or a combination thereof.

According to another example (“Example 14”) further to any one of preceding Examples 1 to 13, the cultivation system comprises a plurality of spores, germinated spores, or both spores and germinated retained by the microstructure of the cultivation substrate.

According to another example (“Example 15”) further to any one of preceding Examples 1 to 14, the cultivation substrate includes a fibrillated material having a microstructure including a plurality of fibrils defining an average inter-fibril distance.

According to another example (“Example 16”) further to any one of preceding Examples 1 to 15, the microstructure of the cultivation substrate is configured to retain spores having an average spore size of up to and including 200 μm.

According to another example (“Example 17”) further to any one of preceding Examples 1 to 16, the spores comprise algal spores.

According to another example (“Example 18”) further to any one of preceding Examples 1 to 16, the spores comprise fungal spores.

According to another example (“Example 19”) further to any one of preceding Examples 1 to 16, the spores comprise plant spores.

According to another example (“Example 20”) further to any one of preceding Examples 1 to 16, the spores comprise bacterial spores.

According to another example (“Example 21”) further to any one of preceding Examples 1 to 20, the cultivation substrate comprises a material having an average density from 0.1 to 1.0 g/cm³.

According to another example (“Example 22”) further to Example 21, the cultivation substrate includes a growth medium comprising the material, and a ratio of the average inter-fibril distance (μm) to the average density (g/cm³) of the fibrillated material is from 1 to 2000.

According to another example (“Example 23”) further to any one of preceding Examples 1 to 22, the cultivation substrate is configured as a fiber, a membrane, a woven article, a non-woven article, a braided article, a knit article, a fabric, a particulate dispersion, or combinations of two or more of the foregoing.

According to another example (“Example 24”) further to any one of preceding Examples 1 to 23, the cultivation substrate includes at least one of a backer layer, a carrier layer, a laminate of a plurality of layers, a composite material, or combinations thereof.

According to another example (“Example 25”) further to any one of preceding Examples 1 to 24, at least a portion of the cultivation substrate is hydrophilic.

According to another example (“Example 26”) further to any one of preceding Examples 1 to 25, at least a portion of the cultivation substrate is hydrophobic.

According to another example (“Example 27”) further to any one of preceding Examples 1 to 26, one or more portions of the cultivation substrate is hydrophobic and one or more portions of the cultivation system is hydrophilic such that the cultivation system is configured to selectively encourage spore retention in the one or more hydrophilic portions of the cultivation substrate.

According to another example (“Example 28”) further to any one of preceding Examples 1 to 27, the cultivation system includes a bioactive agent associated with the cultivation substrate.

According to another example (“Example 29”) further to any one of preceding Examples 1 to 28, the cultivation system an adhesive applied to a surface of the cultivation substrate, imbibed within the microstructure of the cultivation substrate, or both applied to a surface of the cultivation substrate and imbibed within the microstructure of the cultivation substrate.

According to another example (“Example 30”) further to any one of preceding Examples 1 to 29, the cultivation system includes a salt associated with the microstructure of the cultivation substrate.

According to another example (“Example 31”) further to preceding Example 30, the salt is sodium chloride (NaCl).

According to another example (“Example 32”) further to any one of preceding Examples 1 to 31, the cultivation substrate includes a pattern of higher density portions and lower density portions, the lower density portions corresponding to a portion of the cultivation substrate configured to retain spores at least partially within the microstructure of the cultivation substrate.

According to another example (“Example 33”) further to preceding Example 32, the lower density areas are characterized by a density of 1 g/cm³ or less and the higher density portions are characterized by a density of 1.7 g/cm³ or more.

According to another example (“Example 34”) further to any one of preceding Examples 1 to 33 the microstructure includes a pattern of higher porosity portions and lower porosity portions, the lower porosity portions corresponding to a portion of the microstructure configured to retain spores within the microstructure of the cultivations substrate.

According to another example (“Example 35”) further to any one of preceding Examples 1 to 33, the cultivation substrate includes a pattern of higher porosity portions and lower porosity portions, the higher porosity portions corresponding to a portion of the cultivation substrate configured to retain spores within the microstructure of the cultivation substrate.

According to another example (“Example 36”) further to any one of preceding Examples 1 to 35, the cultivation substrate includes a pattern of greater inter-fibril distance portions and lower inter-fibril distance portions, the lower inter-fibril distance portions corresponding to the portion of the cultivation substrate configured to retain spores within the microstructure of the cultivation substrate.

According to another example (“Example 37”) further to any one of preceding Examples 1 to 35, the cultivation substrate includes a pattern of greater inter-fibril distance portions and lower inter-fibril distance portions, the greater inter-fibril distance portions corresponding to the portion of the cultivation substrate configured to retain spores within the microstructure of the cultivation substrate.

According to another example (“Example 38”) further to any one of preceding Examples 32 to 37, the pattern is an organized or selective pattern.

According to another example (“Example 39”) further to any one of preceding Examples 32 to 37, the pattern is a random pattern.

According to another example (“Example 40”) further to any one of preceding Examples 1 to 39, the microstructure is initially in a first retention phase to retain the spores and subsequently in a second growth phase to induce ingrowth of sporelings from the spores on and/or into the microstructure to mechanically couple the sporelings to the microstructure.

According to another example (“Example 41”) further to any one of preceding Examples 1 to 40, nutrients are configured to be delivered via sterile seawater.

According to another example (“Example 42”) further to any one of preceding Examples 1 to 41, the microstructure is configured to irremovably anchor a portion of each of the spores.

According to another example (“Example 43”) further to any one of preceding Examples 1 to 42, the microstructure is configured to irremovably anchor germinated spores.

According to another example (“Example 44”) further to any one of preceding Examples 1 to 43, the cultivation substrate is provided by a plurality of particles in a dispersion formulated for deposition onto a backer layer or carrier substrate.

According to another example (“Example 45”) further to any one of preceding Examples 1 to 44, the cultivation substrate comprises an expanded fluoropolymer.

According to another example (“Example 46”) further to any one of preceding Examples 5 to 45, the cultivation substrate comprises an expanded fluoropolymer wherein the nutrient phase is co-blended with the expanded fluoropolymer.

According to another example (“Example 47”) further to Example 45 or 46, the expanded fluoropolymer is one of: expanded fluorinated ethylene propylene (eFEP), porous perfluoroalkoxy alkane (PFA), expanded ethylene tetrafluoroethylene (eETFE), expanded vinylidene fluoride co-tetrafluoroethylene or trifluoroethylene polymer (eVDF-co-(TFE or TrFE)), and expanded polytetrafluoroethylene (ePTFE).

According to another example (“Example 48”) further to any one of preceding Examples 1 to 44, the cultivation substrate comprises an expanded thermoplastic polymer.

According to another example (“Example 49”) further to preceding Example 48, the expanded thermoplastic polymer is one of: expanded polyester sulfone (ePES), expanded ultra-high-molecular-weight polyethylene (eUHMWPE), expanded polylactic acid (ePLA), and expanded polyethylene (ePE).

According to another example (“Example 50”) further to any one of preceding Examples 1 to 44, the cultivation substrate comprises an expanded polymer.

According to another example (“Example 51”) further to any one of preceding Examples 5 to 44 and 53 the cultivation substrate comprises an expanded polymer wherein the nutrient phase is co-blended with the expanded polymer.

According to another example (“Example 52”) further to any one of preceding Examples 50 or 51, the expanded polymer is expanded polyurethane (ePU).

According to another example (“Example 53”) further to any one of preceding Examples 1-44, the cultivation substrate comprises a polymer formed by expanded chemical vapor deposition (CVD)

According to another example (“Example 54”) further to Example 53, the polymer formed by expanded CVD is expanded polyparaxylylene (ePPX).

The foregoing Examples are just that, and should not be read to limit or otherwise narrow the scope of any of the inventive concepts otherwise provided by the instant disclosure. While multiple Examples are disclosed, still other embodiments will become apparent to those skilled in the art from the following detailed description, which shows and describes illustrative Examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a scanning electron microscopy (SEM) micrograph depicting a microstructure of a cultivation substrate in accordance with some embodiments.

FIG. 2 is an SEM micrograph depicting the microstructure pictured in FIG. 1, but at a higher magnification.

FIG. 3 is an SEM micrograph depicting a microstructure of a cultivation substrate in accordance with some embodiments.

FIG. 4 is an SEM micrograph depicting the microstructure pictured in FIG. 3, but at a higher magnification.

FIG. 5 is a schematic illustration depicting a microstructure of a cultivation substrate in accordance with some embodiments.

FIG. 6 is the micrograph of FIG. 2 with cartoon representations of spores of either 10 μm or 30 μm in diameter overlaid thereon in inter-fibril spaces in accordance with some embodiments.

FIG. 7A is a cross-sectional SEM micrograph depicting ingrowth of dulse seaweed into a microstructure of a cultivation substrate in accordance with some embodiments.

FIG. 7B is a cross-sectional SEM micrograph depicting the ingrowth pictured in FIG. 7A, but at a higher magnification.

FIG. 7C is a cross-sectional optical fluorescence microscopy micrograph depicting ingrowth of dulse seaweed into a microstructure of a cultivation substrate in accordance with some embodiments.

FIG. 8 presents a surface SEM micrograph (top panel) depicting a microstructure of a cultivation substrate prior to seeding with sugar kelp spores in accordance with some embodiments, and an optical fluorescence microscopy micrograph (bottom panel) depicting the cultivation substrate following seeding with sugar kelp spores and germination thereof.

FIG. 9 presents two surface SEM micrographs taken at different magnifications depicting juvenile dulse ingrowth into a microstructure in accordance with some embodiments.

FIG. 10 is a surface optical fluorescence microscopy micrograph depicting ingrowth of dulse seaweed into a microstructure of a cultivation substrate in accordance with some embodiments.

FIG. 11 is an SEM micrograph depicting the superficial surface attachment of developing seaweed to the surface fibers of a high-density material in accordance with some embodiments.

FIG. 12 is an SEM micrograph depicting a woven cultivation substrate in accordance with some embodiments.

FIG. 13 is an SEM micrograph depicting a commercially available porous polyethylene.

FIG. 14 is a collection of photographs depicting growth of dulse on a gel processed polyethylene membrane in accordance with some embodiments (Membrane 1), and a commercially available porous polyethylene (Membrane 2).

FIG. 15 is a collection of photographs depicting growth of kelp on a gel processed polyethylene membrane in accordance with some embodiments (Membrane 1), and a commercially available porous polyethylene (Membrane 2).

FIG. 16 is a photograph depicting growth of dulse on a patterned membrane in accordance with some embodiments.

FIG. 17 photograph depicting growth of kelp on a patterned membrane in accordance with some embodiments.

FIG. 18 is a photograph depicting juvenile sugar kelp sporophyte attachment to a membrane in accordance with some embodiments.

Persons skilled in the art will readily appreciate the accompanying drawing figures referred to herein are not necessarily drawn to scale, but may be exaggerated or represented schematically to illustrate various aspects of the present disclosure, and in that regard, the drawing figures should not be construed as limiting.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

Certain terminology is used herein for convenience only. For example, words such as “top”, “bottom”, “upper,” “lower,” “left,” “right,” “horizontal,” “vertical,” “upward,” and “downward” merely describe the configuration shown in the figures or the orientation of a part in the installed position. Indeed, the referenced components may be oriented in any direction. Similarly, throughout this disclosure, where a process or method is shown or described, the method may be performed in any order or simultaneously, unless it is clear from the context that the method depends on certain actions being performed first.

A coordinate system is presented in the Figures and referenced in the description in which the “Y” axis corresponds to a vertical direction, the “X” axis corresponds to a horizontal or lateral direction, and the “Z” axis corresponds to the interior/exterior direction.

### Description of Various Embodiments

The present disclosure relates to cultivation systems that include a cultivation substrate. The cultivation substrate is used for retention, culture, and/or growth of spores (e.g., for retaining and maintaining algal spores and growing mature seaweed therefrom), and related methods and apparatuses. In various examples, the cultivation system is operable to grow multi-cellular organisms (e.g., seaweed). In some embodiments, the cultivation system is operable to grow multi-cellular organisms in an open-water environment.

Cultivation systems according to the instant disclosure can be used in a variety of applications, including spore capture, spore culture and growth, and spore and/or gametophyte/sporophyte transport and deposition. In certain embodiments, the cultivation substrates described herein can be used as an improved growth substrate for the growth and cultivation of seaweed forms (e.g., spores, gametophytes, sporophytes), resulting in improved yield and throughput relative to current cultivation practices

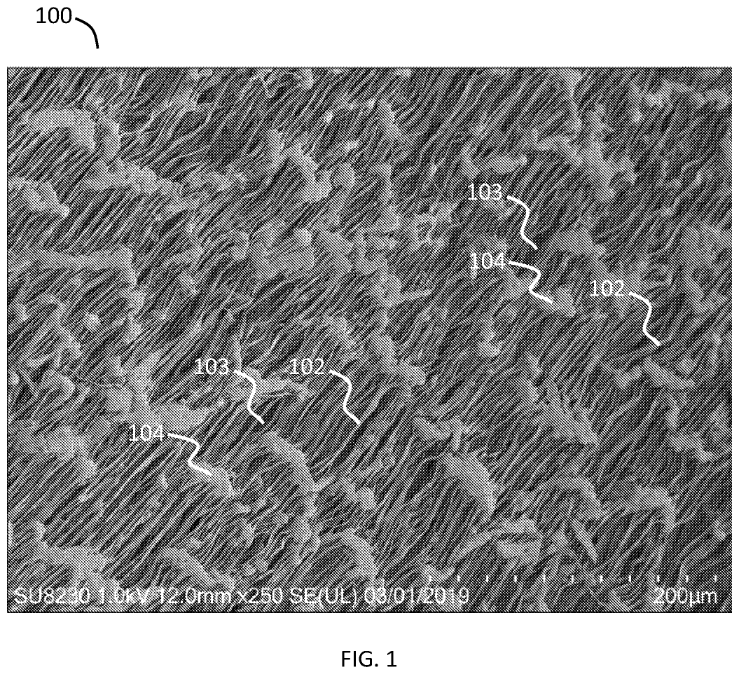

In some embodiments, the cultivation system includes a cultivation substrate which itself includes a fibrillated material having a microstructure including a plurality of fibrils defining an average inter-fibril distance. FIG. 1 is an SEM micrograph depicting a microstructure 100 of a cultivation substrate including a fibrillated material according to some embodiments. The fibrillated material depicted in FIG. 1 having the microstructure 100 is expanded polytetrafluoroethylene (ePTFE). As depicted, the microstructure 100 is defined by a plurality of fibrils 102 that interconnect nodes 104. The fibrils 102 define inter-fibril spaces 103.

The fibrils 102 have a defined average inter-fibril distance, which in some embodiments may be from about 1 μm to about 200 μm, from about 1 μm to about 50 μm, from about 1 μm to about 20 μm, from about 1 μm to about 10 μm, from about 1 μm to about 5 μm, from about 5 μm to about 50 μm, from about 5 μm to about 20 μm, from about 5 μm to about 10 μm, from about 10 μm to about 100 μm, from about 10 μm to about 75 μm, from about 10 μm to about 50 μm, from about 10 μm to about 25 μm, from about 25 μm to about 200 μm, from about 25 μm to about 150 μm, from about 25 μm to about 100 μm, from about 25 μm to about 50, from about 50 μm to about 200 μm, from about 50 μm to about 150 μm, from about 50 μm to about 100 μm, from about 100 μm to about 200 μm, from about 100 μm to about 150 μm, or from about 150 μm to about 200 μm. In some embodiments, the fibrils 102 may have an average inter-fibril distance of about 1 μm, about 2 μm, about 3 μm, about 4 μm, about 5 μm, about 10 μm, about 20 μm, about 30 μm, about 40 μm, about 50 μm, about 60 μm, about 70 μm, about 80 μm, about 90 μm, about 100 μm, about 110, about 120 μm, about 130 μm, about 140 μm, about 150 μm, about 160 μm, about 170 μm, about 180 μm, about 190 μm, or about 200 μm.

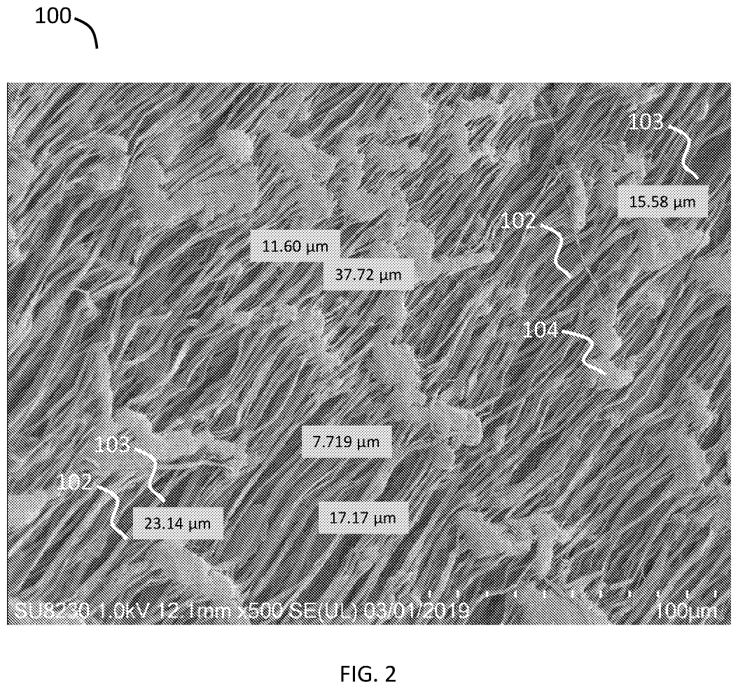

FIG. 2 is a higher magnification SEM micrograph of the microstructure depicted in FIG. 1. FIG. 2 identifies the dimension of select inter-fibril spaces 103 in μm.

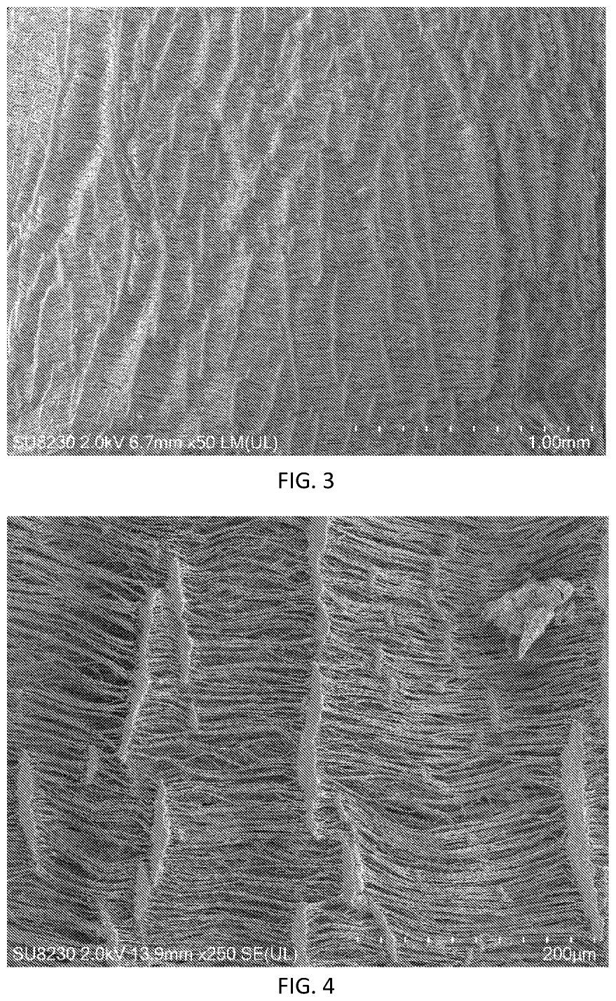

FIG. 3 is an SEM micrograph depicting another microstructure of a cultivation substrate that includes a fibrillated ePTFE material according to some embodiments.

FIG. 4 is a higher magnification SEM micrograph of the microstructure depicted in FIG. 3.

At least some of the fibrils 102 are sufficiently spaced from each other to retain a spore in an inter-fibril space 103.

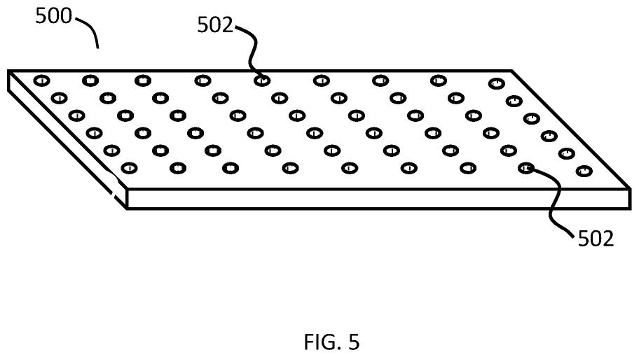

FIG. 5 is a perspective view of a schematic representation of the microstructure of a cultivation substrate according to some embodiments. As depicted, the microstructure 500 is defined by a plurality of pores 502.

The pores 502 may be round, approximately round, or oblong. The pores 502 may have a diameter or approximate diameter from about 1 μm to about 200 μm, from about 1 μm to about 50 μm, from about 1 μm to about 20 μm, from about 1 μm to about 10 μm, from about 1 μm to about 5 μm, from about 5 μm to about 50 μm, from about 5 μm to about 20 μm, from about 5 μm to about 10 μm, from about 10 μm to about 100 μm, from about 10 μm to about 75 μm, from about 10 μm to about 50 μm, from about 10 μm to about 25 μm, from about 25 μm to about 200 μm, from about 25 μm to about 150 μm, from about 25 μm to about 100 μm, from about 25 μm to about 50, from about 50 μm to about 200 μm, from about 50 μm to about 150 μm, from about 50 μm to about 100 μm, from about 100 μm to about 200 μm, from about 100 μm to about 150 μm, or from about 150 μm to about 200 μm. In some embodiments, the pores 502 may have a diameter or approximate diameter of about 1 μm, about 2 μm, about 3 μm, about 4 μm, about 5 μm, about 10 μm, about 20 μm, about 30 μm, about 40 μm, about 50 μm, about 60 μm, about 70 μm, about 80 μm, about 90 μm, about 100 μm, about 110, about 120 μm, about 130 μm, about 140 μm, about 150 μm, about 160 μm, about 170 μm, about 180 μm, about 190 μm, or about 200 μm.

In some embodiments, the inter-fibril spaces 103 of FIG. 1 form the pores 502 of FIG. 5. That is, a microstructure 100 having a plurality of fibrils 102 may form the porous microstructure 500. However, not all microstructures 500 having pores 502 are fibrillated.

The microstructure of the cultivation substrate is configured to retain spores and sporophytes, gametophytes, or other organisms grown from the retained spores. In some embodiments, the microstructure is configured to retain algal spores, algal sporophytes and/or gametophytes, plant spores, seedlings, bacterial endospores, fungal spores, or a combination thereof. In some embodiments, the cultivation substrate retains a plurality of spores and/or organisms grown therefrom (e.g., sporophytes and/or gametophytes). The plurality of spores and/or organisms may all be of the same type, or of two or more different types. In some embodiments, the cultivation substrate retains two different spore types that display a symbiotic relationship when cultured or grown together. For sake of simplicity, throughout this disclosure reference will be made to “spores,” although gametophytes, sporophytes, seedlings, or other organisms grown from the spores are also contemplated by this term and are considered to be within the purview of the disclosure.

In some embodiments, in addition to retaining spores, cultivation systems and substrates of the instant disclosure promote germination of and growth from the retained spores. That is, the cultivation systems and substrates viably maintain the retained spores. In certain embodiments, the microstructure is configured to irremovably anchor at least a portion of a spore.

The cultivation substrate, for example, creates a microenvironment conducive to the germination of and growth from the retained spores. In some embodiments, the microstructure is initially in a first retention phase, where the microstructure functions to retain and maintain the target spore. The microstructure subsequently is in a second growth phase, where germination of the spore is induced, and ingrowth of sporelings (e.g., sporophytes, gametophytes, seedlings, etc.) from the spore on and/or into the microstructure, thereby resulting in a mechanical coupling, or anchoring, of the sporelings to the microstructure. Thus, in some embodiments, the microstructure is configured to irremovably anchor germinated spores, preventing loss of the germinated spores during, for example, transport or placement in the field (e.g., an open-water environment), or loss to environmental factors (e.g., currents).

In certain embodiments, the cultivation substrate creates a selective microenvironment conducive to the germination of and growth from a target spore while inhibiting or preventing germination, growth, and/or proliferation of non-target spores or other cells. A selective microenvironment can be achieved by, for example, providing a combination of inter-fibril distance and/or pore size, material density, ratio of inter-fibril distance to average density of material, depth or thickness, hydrophobicity, and presence or absence of nutrient sources, moisture, bioactive agents, and adhesives that supports germination of and growth from the target spore while inhibiting or preventing germination, growth, and/or proliferation of non-target spores or other cells.

Several factors may affect retention and/or viable maintenance of the spores and organisms grown therefrom. Such factors include, for example, the inter-fibril distance and/or pore size, material density, a ratio of inter-fibril distance to average density of material, depth or thickness, hydrophobicity, and presence or absence of nutrient sources, moisture, bioactive agents, and adhesives. These factors will each be described in more detail.

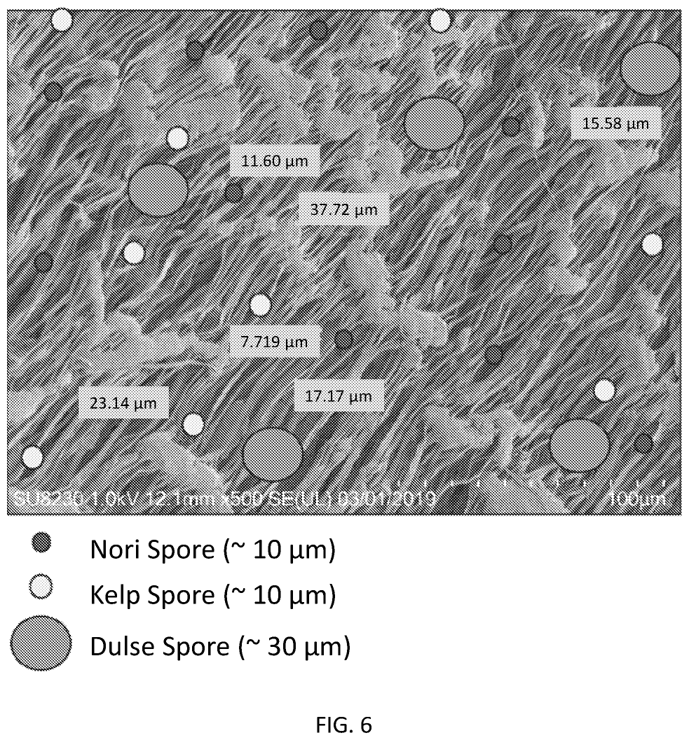

The distance between two fibrils (i.e., inter-fibril distance) defines an inter-fibril space 103. In some embodiments, an inter-fibril space 103—and thus the inter-fibril distance—is sufficient to retain a spore therein; the spore is retained between the two fibrils defining the inter-fibril space. The inter-fibril distance is sufficient to allow at least a portion of the spore to enter between the two fibrils defining the inter-fibril space 103. In some embodiments, the spore is thereby retained within the microstructure of the cultivation substrate. FIG. 6 is a modified version of the photograph of FIG. 2, depicting a microstructure of a cultivation substrate including a fibrillated material and overlaid with representative spores having a diameter of either about 10 μm (e.g., nori and kelp spores) or about 30 μm (e.g., dulse spores). FIG. 6 illustrates how and where target spores may enter between the two fibrils defining an inter-fibril space.

In some embodiments, the average inter-fibril distance is controlled in order to encourage ingress of at least portions of spores into the microstructure. For example, where it is desirous for the microstructure to retain spores of dulse (Palmaria palmata), which have a diameter of about 30 μm, the average inter-fibril distance of the microstructure is about 30 μm, or slightly larger (e.g., about 32 μm to about 35 μm). Where it is desirous for the microstructure to retain spores of nori or kelp, which each have a spore having a diameter of about 10 μm, the average inter-fibril distance of the microstructure is about 10 μm, or slightly larger (e.g., about 12 μm to about 15 μm). In some embodiments, it may be desirous to retain spores of multiple species (e.g., dulse, nori, and kelp). In such embodiments, the average inter-fibril distance is sufficient to allow at least a portion of the spores of the multiple species to enter the inter-fibril space and be retained there. In some embodiments, target spores have a diameter of about 0.5 μm to about 200 μm.

In some embodiments, about half of the target spore may enter the inter-fibril space 103. In such embodiments, the inter-fibril distance is at least equal to a dimension (e.g., diameter or width) of the target spore. In some embodiments, the inter-fibril distance is slightly larger than the dimension of the target spore. This allows for the entire spore to enter the inter-fibril space 103 and be retained therein.

In some embodiments, more than half of the target spore may enter the inter-fibril space 103, up to the entire spore. In such embodiments, the portion of the spore entering the inter-fibril space 103 may be governed by the depth of a pore, the opening of which is defined by the inter-fibril space. The depth of the pore may be controlled by, for example, material density.

In some embodiments, only a portion of the spore enters the inter-fibril space 103. Therefore, in instances where the inter-fibril distance is less than the diameter of the target spore, the target spore may only partially enter the inter-fibril space 103. Where the target spore only partially enters the inter-fibril space 103, the target spore may none-the-less be retained therein if a sufficient portion of the target spore enters the inter-fibril space 103. In some embodiments, a substance such as an adhesive applied to the microstructure may reduce the portion of the spore required to enter the inter-fibril space 103 and aid in retention.

In some embodiments, the microstructure is formed by a non-fibrillated material. In certain embodiments, the pore openings 502 are inherent to the material of the cultivation substrate. It will be recognized that different materials may have different pore opening properties, and that a material may be manufactured or otherwise manipulated to provide the desired pore opening properties. In other embodiments, the pore openings 502 are formed by micro drilling techniques such as, for example: mechanical micro drilling, such as ultrasonic drilling, powder blasting or abrasive water jet machining (AWJM); thermal micro drilling, such as laser machining; chemical micro drilling, including wet etching, deep reactive ion etching (DRIE) or plasma etching; and hybrid micro drilling techniques, such as spark-assisted chemical engraving (SACE), vibration-assisted micromachining, laser-induced plasma micromachining (LIPMM), and water-assisted micromachining.

In those embodiments where the microstructure is formed by a non-fibrillated material, the pore openings 502 act much like the inter-fibril spaces 103 described and are of a sufficient size to allow at least a portion of a target spore to enter the pore opening 502. In some embodiments, the spore is thereby retained within the microstructure of the cultivation substrate. In some embodiments, the size of pore openings 502 is controlled to encourage ingress of a least portions of target spores into the microstructure. For example, where it is desirous for the microstructure to retain spores of dulse (Palmaria palmata), which have a diameter of about 30 μm, the pore openings 502 of the microstructure have a diameter of about 30 μm, or slightly larger (e.g., about 32 μm to about 35 μm). In some embodiments, target spores have a diameter of about 0.5 μm to about 200 μm.

In some embodiments, about half of the target spore may enter the pore opening 502. In such embodiments, the pore opening is at least equal to a dimension (e.g., diameter or width) of the target spore. In some embodiments, the pore opening is slightly larger than the dimension of the target spore. This allows for the entire spore to enter the pore opening 502 and be retained therein.

In some embodiments, more than half of the target spore may enter the pore opening 502, up to the entire spore. In such embodiments, the portion of the spore entering the pore opening 502 may be governed by the pore depth. The depth of the pore may be controlled by, for example, material density.

In some embodiments, only a portion of the spore enters the pore opening 502. Therefore, where the pore opening is smaller than the diameter of the target spore, the target spore may only partially enter the pore opening 502. Where the target spore only partially enters the pore opening 502, the target spore may none-the-less be retained therein when a sufficient portion of the target spore enters the pore opening. In some embodiments, a substance such as an adhesive applied to the microstructure may reduce the portion of the spore required to enter the pore opening 302 and aid in retention.

In some embodiments, the cultivation substrate includes a low-density material. The low-density material may be fibrillated or non-fibrillated, and in some embodiments, defines the microstructure of the cultivation substrate. The density of the low-density material may be about 0.1 g/cm³, about 0.2 g/cm³, about 0.3 g/cm³, about 0.4 g/cm³, about 0.5 g/cm³, about 0.6 g/cm³, about 0.7 g/cm³, about 0.8 g/cm³, about 0.9 g/cm³, or about 1.0 g/cm³. In some embodiments, the density of the low-density material is from about 0.1 g/cm³ to about 1 g/cm³.

In some embodiments, the low-density material provides a sufficient pore depth to retain spores in inter-fibril spaces 103 or pore openings 502.

In some embodiments, the dimensions of the pore openings (length (μm) and width (μm)), whether formed by a fibrillated or non-fibrillated material, together with the depth at which target spores enter the pores (μm) define a capture ratio. Each spore type may have a different capture ratio required for adequate retention of spores by the microstructure. The required capture ratio may be influenced by the properties of the material making up the microstructure and the presence or absence of nutrients, adhesives, and/or bioactive agents.

In some embodiments, the low-density material allows the spore to germinate and grow into the low-density material. For example, as dulse spores retained in a low-density material having a microstructure described herein develop into gametophytes and then sporophytes, the dulse grows into the low-density material in all three dimensions (i.e., horizontally in x- and y-dimensions and depth-wise in the z-dimension). This three-dimensional growth allows for improved retention of the dulse gametophytes and sporophytes.

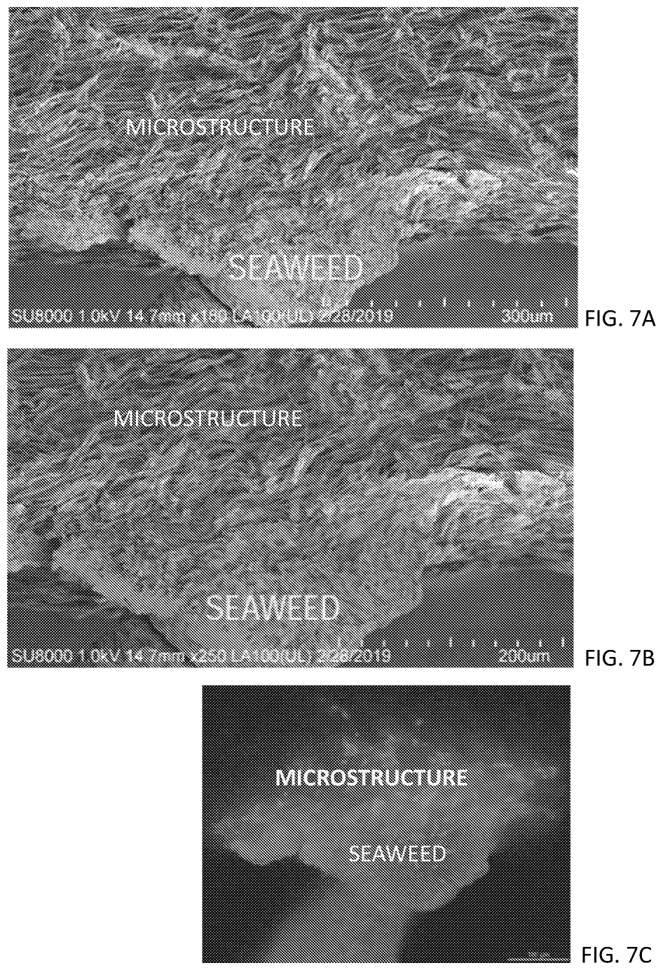

FIGS. 7A and 7B are cross-sectional SEM micrographs taken at two different magnifications of a low-density microstructured material according to some embodiments, depicting dulse seaweed three-dimensional ingrowth into the low-density material. FIG. 7C is a cross-sectional micrograph generated using optical fluorescence microscopy depicting dulse seaweed ingrowth into the low-density material.

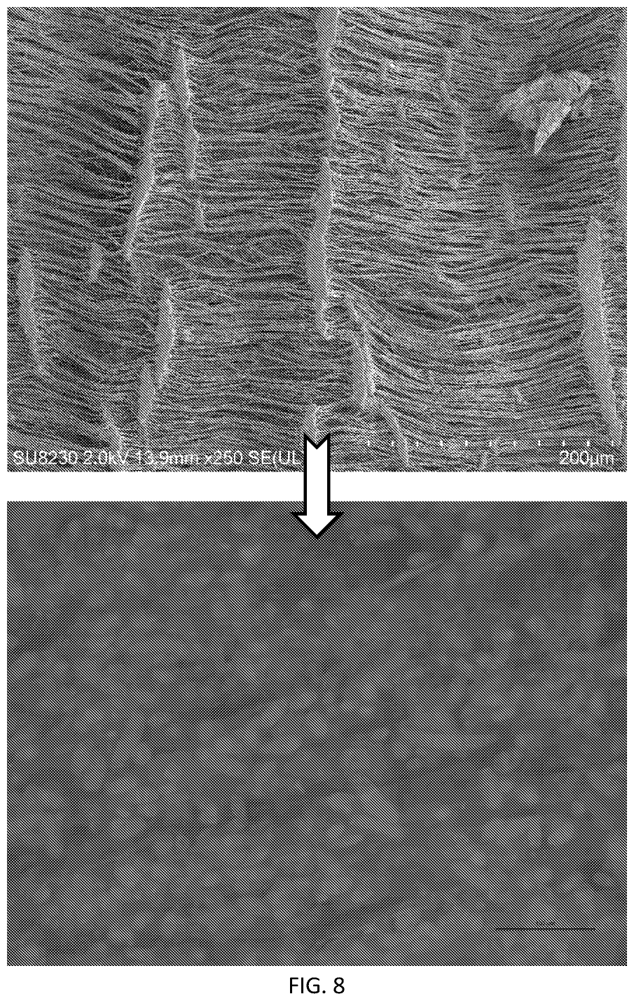

FIG. 8 (top panel) is an SEM micrograph of the surface of a low density microstructured material according to some embodiments. FIG. 8 (bottom panel) depicts the same cultivation substrate material as the top panel following seeding with sugar kelp spores and germination thereof.

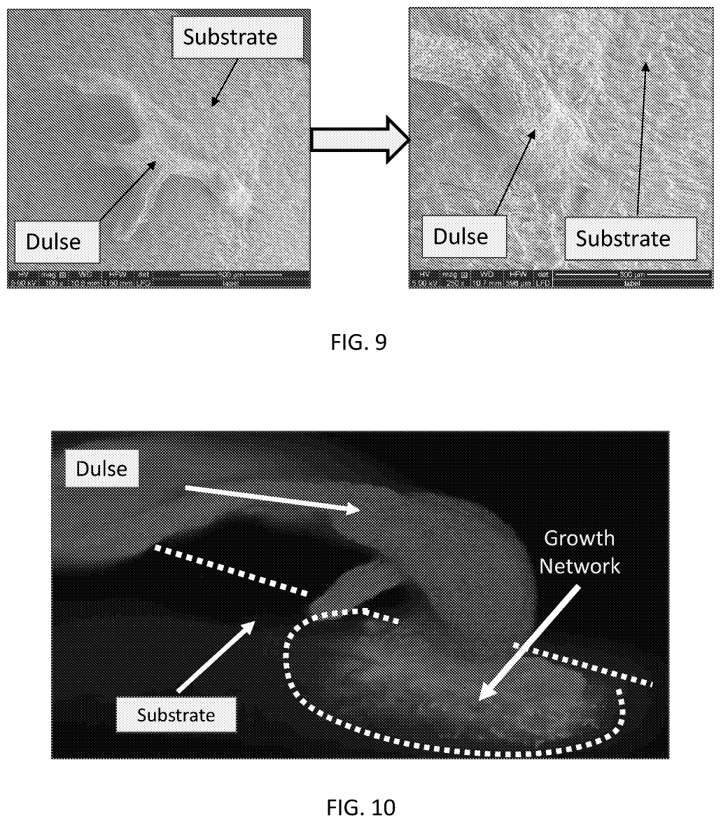

FIG. 9 depicts SEM micrographs of the surface of a microstructure taken at two different magnifications, where dulse seaweed can clearly be seen to be attached to and growing into the microstructure. FIG. 10 depicts a fluorescence microscopy micrograph of the surface of a microstructure to which the dulse seaweed is attached and growing into the microstructure. The seaweed growth is observed to be growing into the microstructure in a ‘growth network’ in all three dimensions.

It is evident from the micrographs of FIG. 7A-FIG. 10 that the dulse seaweed is able to grow into the microstructure of the fibrillated ePTFE in all three dimensions, securely anchoring the seaweed within the microstructure.

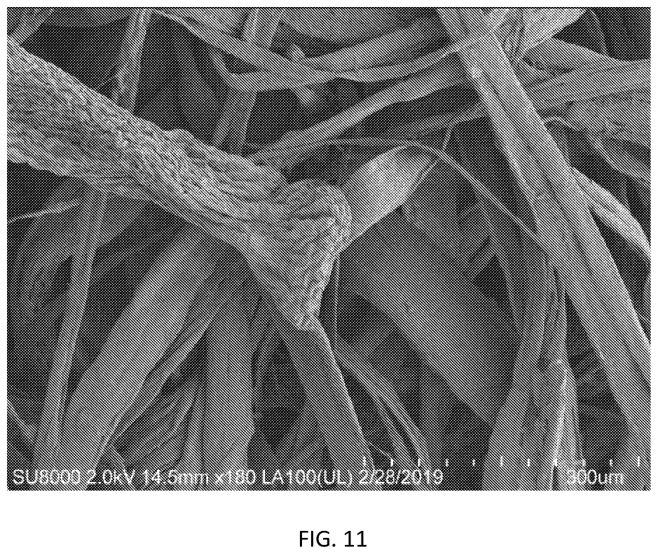

Conversely, FIG. 11 is a micrograph depicting dulse seaweed growing on the surface of a higher-density fibrillated material. The growing dulse is unable to grow into the higher-density material, and rather attaches solely to the fibrils at the material's surface. This results in weaker retention of the dulse gametophyte relative to the low-density material, in which the developing dulse gametophyte becomes anchored.

In some embodiments, germinated spores grow deep into the microstructure. This deep ingrowth and incorporation into the microstructure gives additional benefits in protecting the germinated spores from external environments (e.g., in the case of seaweed gametophytes, the sea and its currents). In some embodiments, the depth of penetration of the germinated spores relative to the initial size of the spore is from about 1:1 to about 200:1. For example, for a dulse spore having an initial diameter of about 30 μm, the dulse sporophyte may grow into the microstructure to a depth of about 30 μm to about 6 mm.

In some embodiments, the low-density material has a thickness sufficient to allow for a desired level of ingrowth. In some embodiments, the cultivation substrate includes a single layer of the low-density material. In some embodiments, the cultivation substrate includes two or more layers of the low-density material. In certain embodiments, the two or more layers are present in a laminate, i.e., a laminate of a plurality of layers of the low-density material.

In some embodiments, the inter-fibril distance and the density of the material having a microstructure defines a ratio of the average inter-fibril distance (μm) to the average density (g/cm³) of the fibrillated material. In some embodiments, the ratio of the average inter-fibril distance (μm) to the average density (g/cm³) of the fibrillated material may be about 1:1, about 10:1, about 20:1, about 30:1, about 40:1, about 50:1, about 60:1, about 70:1, about 80:1, about 90:1, about 100:1, about 125:1, about 150:1, about 175:1, about 200:1, about 225:1, about 250:1, about 275:1, about 300:1, about 325:1, about 350:1, about 375:1, about 400:1, about 425:1, about 450:1, about 475:1, about 500:1, about 550:1, about 600:1, about 650:1, about 700:1, about 750:1, about 800:1, about 900:1, about 1000:1, about 1250:1, about 1500:1, about 1750:1, or about 2000:1. In some embodiments, the ratio of the average inter-fibril distance (μm) to the average density (g/cm³) of the fibrillated material is from about 1:1 to about 2000:1.

In some embodiments, the cultivation substrate includes one or more adhesives. An adhesive may be applied to the surface of the microstructure, imbibed within the microstructure, or both applied to the surface and imbibed within the microstructure. In some embodiments, the adhesive includes one or more cell-adhesive ligands specific to the spore(s) to be retained by the cultivation substrate.

In some embodiments, a cultivation substrate described herein includes a nutrient phase associated with at least a portion of the cultivation substrate. The nutrient phase serves to viably maintain the spores and germinated spores retained by the cultivation substrate. In some embodiments, the nutrient phase promotes germination of and growth from the retained spores within the microstructure. In some embodiments, the nutrient phase acts to maintain and/or encourage attachment to and ingrowth into or integration within the microstructure.

In some embodiments, the nutrient phase acts as a chemoattractant capable of attracting the spores to predetermined locations of the cultivation substrate to which the nutrient phase is applied or included.

The nutrient phase can be located within the microstructure of the cultivation substrate, on the microstructure (e.g., on its surface), or located both within and on the microstructure. In some embodiments, the nutrient phase is applied to a surface of the cultivation substrate as a coating. In some embodiments, the nutrient phase is included within the material forming the microstructure. Where the nutrient phase is included within the material forming the microstructure, the nutrient phase may encourage ingrowth into or integration within the microstructure.

In some embodiments, the nutrient phase includes at least one nutrient beneficial to the target spore and resulting germinated spore to be retained by the cultivation substrate. For example, where spores are to be retained by the microstructure, the nutrient phase can include macronutrients (e.g., nitrogen, phosphorous, carbon, etc.), micronutrients (e.g., iron, zinc, copper, manganese, molybdenum, etc.), and vitamins (e.g., vitamin B12, thiamine, biotin) that will support the growth and health of the germinated dulse spore. The nutrients of the nutrient phase can be provided in various forms. For example, nitrogen can be provided as ammonium nitrate (NH₄NO₃), ammonium sulfate ((NH₄)₂SO₄), calcium nitrate (Ca(NO₃)₂), potassium nitrate (KNO₃), urea (CO(NH₂)₂), etc. It will be recognized by those of skill in the art which nutrients would be beneficial to include in the nutrient phase so as to viably maintain the spores and resulting germinated spores to be retained by the cultivation substrate.

Which nutrients to include in the nutrient phase will depend on which spores are to be retained by the cultivation substrate, as various spore types and germinated spores will have different nutrient needs, as well as the intended use of the cultivation system. For example, where a cultivation substrate retaining spores and/or germinated spores is to be introduced into an environment that is deficient in essential nutrients, all nutrients required by the spores/germinated spores can be included in the nutrient phase. Where a cultivation substrate retaining spores/germinated spores is to be introduced into an environment having at least one essential nutrient, those environmentally-available essential nutrients may be excluded from the nutrient phase or included at a lower concentration. The cultivation substrate may also act to concentrate nutrients from the environment by capturing the environmental nutrients in the microstructure. This may be advantageous in environments where environmental nutrients are present only in low concentrations.

In some embodiments, and as further described elsewhere herein, the cultivation system can be used to transport retained spores/germinated spores from location to another. Where the cultivation system functions as a transportation system, the nutrient phase may include sufficient nutrient levels to viably support the retained spores/germinated spores during transport. In some embodiments the nutrient phase may include sufficient nutrient levels to viably maintain the retained spores/germinated spores post-transport, following introduction of the retained spores/germinated spores into a new environment.

In some embodiments the nutrient phase includes one or more carriers. Carriers can include, for example, liquid carriers, gel carriers, and hydrogel carriers. In some embodiments, a carrier of the nutrient phase is an adhesive. Including an adhesive as a carrier of the nutrient phase can function to ensure that the nutrient phase remains on and/or within the cultivation substrate. Where the nutrient phase is applied to a surface of the cultivation substrate and includes an adhesive as a carrier, the nutrient face may also function to promote retention of spores within the microstructure.

In some embodiments, the nutrient phase is formulated to control release rates of the nutrients.

In some embodiments, the cultivation substrate further comprises a salt associated with the microstructure. In some embodiments, the salt is sodium chloride (NaCl). Salt associated with the microstructure can produce and maintain a saline microenvironment for the retained spores/germinated spores. This can be particularly advantageous when seaweed and marine plants are retained by the cultivation substrate. In some embodiments, a saline microenvironment within the cultivation substrate can be maintained when the cultivation substrate is submerged in fresh water, thereby viably maintaining marine species and avoiding the need to maintain a saline culture environment, which can be difficult and costly.

In some embodiments, the cultivation substrate includes a liquid-containing phase associated with at least a portion of the cultivation substrate. The liquid-containing phase serves to provide and maintain moisture within the microstructure's microenvironment, which may be beneficial to the viable maintenance of the spores/germinated spores retained therein.

In some embodiments, the cultivation substrate includes a liquid wicking material. The liquid wicking material can be the same material that forms the microstructure. The liquid wicking material functions to maintain moisture within the microstructure's microenvironment.

While spores and endospores may be viably maintained in an arid environment, the germinated spores will generally require moisture to grow and/or proliferate. By maintaining a moist microenvironment (e.g., by including a liquid-containing substrate and/or a liquid wicking material), it may be possible to transport the culture system having spores/germinated spores retained therein without having to maintain the cultivation system in an aqueous environment.

In some embodiments, the liquid containing phase is entrained within the microstructure, entrained on the microstructure, or entrained both within and on the microstructure. In some embodiments, the liquid containing phase is present as a coating on a surface of the cultivation substrate.

In some embodiments, the liquid containing phase includes, for example, a hydrogel, a slurry, a paste, or a combination of a hydrogel, a slurry, and/or a paste. In some embodiments, the liquid containing phase is a carrier for the nutrient phase.

In some embodiments, at least a portion of the cultivation substrate is hydrophilic. Such hydrophilic portions of the cultivation substrate may contribute to the microstructure's ability to retain the spores.

In some embodiments, at least a portion of the cultivation substrate is hydrophobic. Such hydrophobic portions of the cultivation substrate may reduce or prevent or resist retention of spores. This may help reduce or prevent biofouling and attachment of unwanted spores or other cells.

In some embodiments, one or more portions of the cultivation substrate is hydrophobic and one or more portions of the cultivation substrate is hydrophilic, such that spores are selectively encouraged to be retained in the one or more hydrophilic portions of the cultivation substrate.

In some embodiments, the cultivation substrate may include one or more bioactive agents associated with the cultivation substrate. Bioactive agents include any agent having an effect, whether positive or negative, on the cell or organism coming into contact with the agent. Suitable bioactive agents may include, for example, biocides and serums. Biocides may be associated with portions of the microstructure to prevent attachment and growth of unwanted cells or organisms to those portions of the microstructure. Unwanted cells may include non-target cells such as bacteria, yeast, and algae, for example. Biocides may also deter pests, such as insects. In some embodiments, the biocide prevents attachment and growth of the target spore to portions of the cultivation substrate where attachment and growth is not desired. In some embodiments, serums may be applied to portions of the cultivation substrate. Serums may aid in spore attachment and retention and/or encourage germination of or growth from the spore. Serums may include cell-adhesive ligands, for example, as well as provide a source of growth factors, hormones, and attachment factors.

In some embodiments, the microstructure of the cultivation substrate is patterned. By specifically patterning the microstructure, it is possible to specifically retain target spores at described portions of the microstructure while excluding cells from other portions.

In some embodiments, the microstructure includes a pattern of higher density portions and lower density portions. In such a configuration, the lower density portions correspond to a portion of the microstructure configured to retain and viably maintain the target spores, while the higher density portions inhibit or prevent retention of cells. The density pattern may extend in any dimension. For example, a high-density/low-density pattern may extend in the x- or y-dimension of the cultivation substrate, or in the z-dimension. When extending in the z-dimension, the outermost portion will generally be a lower density portion configured to retain and viably maintain the target spores. Underlying portions may be of a higher density, or may be of an even lower density than the outermost portion. Where the underlying portion is of a higher density, ingrowth of the germinated spores will be inhibited or prevented. Where the underlying portion is of a lower density than the outermost portion, ingrowth of the germinated spores will be encouraged and/or facilitated. In some embodiments, the density pattern or gradient in the z-dimension results from concentric wraps of microstructure material having differing densities, or from a laminate configuration in which each lamina has a different density. In some embodiments, the density pattern can extend in two or all three dimensions. In some embodiments, portions of the microstructure have a density gradient.

Density can be measured in various ways, including, for example, measuring dimensions and weight of the material. In addition, wetting experiments can be conducted to derive density values. Density can be modified by, for example, altering inter-fibril distance, number of fibrils per unit volume, number of pores per unit volume, and pore size.

In some embodiments, the lower density portions are characterized by a material density of about 1.0 g/cm³ or less, whereas the higher density portions are characterized by a density of about 1.7 g/cm³ or greater. As depicted by FIGS. 7A-70 and 11, attachment and retention of germinated spores (dulse seaweed sporophytes depicted) can be significantly affected by microstructure material density, with the lower density material (i.e., about 1.0 g/cm³ or less) demonstrating improved ingrowth and retention.

In some embodiments, the density is that of the material itself that forms the microstructure; i.e., does not have any inclusions such as a nutrient phase, liquid containing phase, etc.

In some embodiments, the density is that of the material and an inclusion such as a nutrient phase, a liquid containing phase, or a density-altering filler. In some embodiments, portions of the microstructure are filled with a filler to alter the density, thereby altering the ability of that portion of the microstructure to retain spores and/or prevent ingrowth into the microstructure.

In some embodiments, the cultivation substrate includes a material having a pattern of higher porosity portions and lower porosity portions. In some embodiments, the lower porosity portions correspond to portions of the microstructure configured to retain and viably maintain the target spores. In some embodiments, the higher porosity portions correspond to portions of the microstructure configured to retain and viably maintain the target spores.

In some embodiments, the cultivation substrate includes a pattern of greater inter-fibril distance portions and lower inter-fibril distance portions. In some embodiments, the lower inter-fibril distance portions correspond to the portions of the microstructure configured to retain and viably maintain the spores. In such embodiments, the higher inter-fibril distance portions have inter-fibril distances too great to retain the target spores. In some embodiments, the higher inter-fibril distance portions correspond to the portions of the microstructure configured to retain and viably maintain the spores. In such embodiments, the lower inter-fibril distance portions have inter-fibril distances too small to retain the target spores.

In some embodiments, the pattern of the patterned cultivation substrate is generated by controlling at least two of density, porosity, and average inter-fibril distance. In some embodiments, the pattern of the patterned cultivation substrate, whether involving density, porosity, average inter-fibril distance, or a combination thereof, may be an organized or selective pattern, or may be a random pattern.

In some embodiments, the pattern can be set or adjusted by selective application of longitudinal tension. Setting or adjusting the pattern by application of longitudinal tension allow for one to alter the pattern mechanically. In some embodiments, a pattern is set or adjusted in fibrillated material by selective application of longitudinal tension.

In some embodiments, a patterned cultivation substrate includes portions that have two or more characteristics favorable to spore retention. For example, a patterned cultivation substrate can have portions of low-density (i.e., about 1.0 g/cm³ or less) and an average inter-fibril distance selected to retain the target spores (e.g., about 30 μm for dulse spores). These same portions may further be hydrophilic and/or include one or more of a nutrient phase, an adhesive, and a bioactive agent. The density, inter-fibril distance, hydrophobicity, nutrient phase, adhesive, and bioactive agent, for example, may each be selected to preferentially retain a target spore.

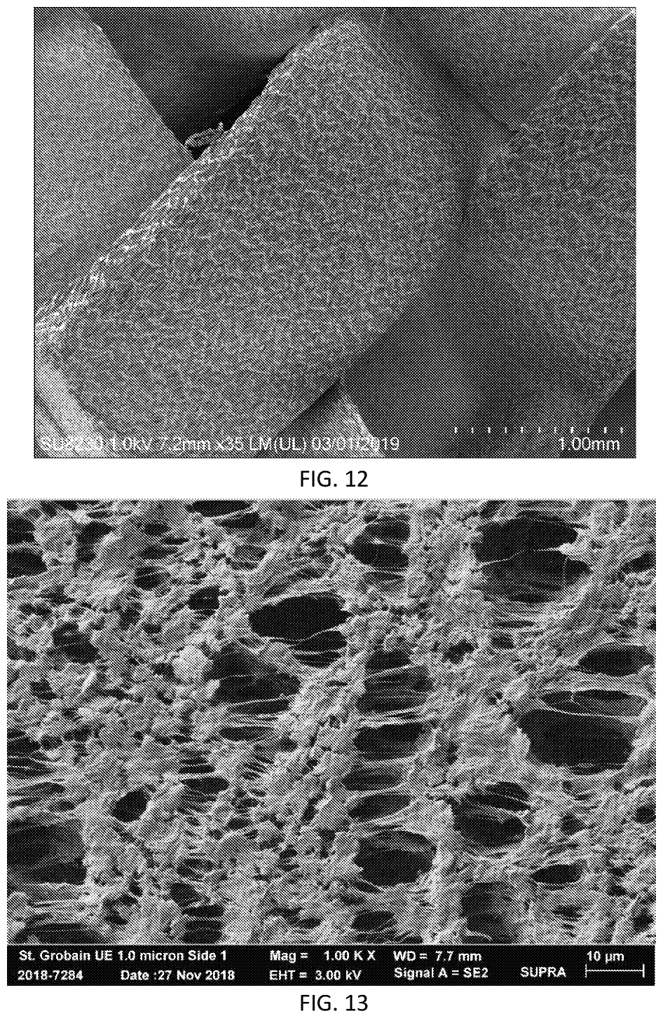

In some embodiments, the cultivation substrate is configured as a fiber, a membrane, a woven article, a non-woven article, a braided article, a fabric, a knit article, a particulate dispersion, or combinations of these. FIG. 12 is a photograph of a cultivation substrate according to certain embodiments, where the cultivation substrate is configured as a woven article. As demonstrated by FIG. 12, each strand of the woven article comprises a microstructure. In such a configuration, not only can target spores be retained and germinated spores grow through the depth of the strand, but can also grow in the spaces between the woven strands. In the case of dulse seaweed, this can provide for additional mechanical retention capacity as the seaweed grows around the woven strands.

In some embodiments, the cultivation system includes at least one of a backer layer, a carrier layer, a laminate of a plurality of layers, a composite material, or combinations of these. The cultivation substrate can be deposited on the backer layer or carrier layer, or included in a laminate. The backer layer can be, for example, a rope or metal cable. For example, where the cultivation substrate retains and viably maintains seaweed spores, the cultivation substrate can be deposited on a rope or metal cable to produce a seed rope, eliminating the need to wrap a seed string around the rope in the field for open water rope cultivation of seaweed.

In some embodiments, the material having the microstructure itself has sufficient strength to be moved as a conveyor belt through various growth stages of the retained spores, including harvest of the germinated spores. In some embodiments, the material having the microstructure is deposited on a backer layer, carrier layer, or formed into a laminate to produce a cultivation system having sufficient strength to be moved as a conveyor belt through various growth stages of the retained spores, including harvest of the germinated spores.

In some embodiments, the cultivation substrate is configured as a particulate dispersion. The microstructure is provided by a plurality of particles in a dispersion formulated for deposition onto a backer layer or a carrier substrate to form the cultivation system. The particles can be, for example, shredded or otherwise fragmented pieces of a fiber, a membrane, a woven article, a non-woven article, a braided article, a fabric, or a knit article having a microstructure as described herein. In some embodiments, spores are contacted with the particles prior to deposition onto a backer layer or carrier substrate. In other embodiments, spores are contacted with the particles following deposition onto the backer layer or carrier substrate. The particulate dispersion may be deposited onto the backer layer or carrier substrate by, for example, spraying, dip-coating, brushing, or other coating means. In embodiments in which spores are retained in the microstructure of the particles prior to deposition, care must be taken to ensure that the deposition method does not negatively affect the retained spores. Spores and endospores may be more resilient and capable of withstanding deposition in such a manner.

In some embodiments, the cultivation substrate comprises an expanded fluoropolymer. In some embodiments, the expanded fluoropolymer forms the microstructure of the cultivation substrate. In some embodiments, the expanded fluoropolymer is selected from the group of expanded fluorinated ethylene propylene (eFEP), porous perfluoroalkoxy alkane (PFA), expanded ethylene tetrafluoroethylene (eETFE), expanded vinylidene fluoride co-tetrafluoroethylene or trifluoroethylene polymer (eVDF-co-(TFE or TrFE)), expanded polytetrafluoroethylene (ePTFE), and modified ePTFE. Examples of suitable expanded fluoropolymers include fluorinated ethylene propylene (FEP), porous perfluoroalkoxy alkane (PFA), polyester sulfone (PES), poly (p-xylylene) (ePPX) as taught in U.S. Patent Publication No. 2016/0032069, ultra-high molecular weight polyethylene (eUHMWPE) as taught in U.S. Pat. No. 9,926,416 to Sbriglia, ethylene tetrafluoroethylene (eETFE) as taught in U.S. Pat. No. 9,932,429 to Sbriglia, polylactic acid (ePLLA) as taught in U.S. Pat. No. 7,932,184 to Sbriglia, et al., vinylidene fluoride-co-tetrafluoroethylene or trifluoroethylene [VDF-co-(TFE or TrFE)] polymers as taught in U.S. Pat. No. 9,441,088 to Sbriglia

In some embodiments, the expanded fluoropolymer includes the nutrient phase. This may be achieved by co-blending the nutrient phase with the fluoropolymer resin prior to extrusion and expansion of the fluoropolymer.

In some embodiments, the cultivation substrate comprises an expanded thermoplastic polymer. In some embodiments, the expanded thermoplastic polymer forms the microstructure of the cultivation substrate. In some embodiments, the expanded thermoplastic polymer is selected from the group of expanded polyester sulfone (ePES), expanded ultra-high-molecular-weight polyethylene (eUHMWPE), expanded polylactic acid (ePLA), and expanded polyethylene (ePE).

In some embodiments, the cultivation substrate comprises an expanded polymer. In some embodiments, the expanded polymer forms the microstructure of the cultivation substrate. In some embodiments, the expanded polymer is expanded polyurethane (ePU).

In some embodiments, the expanded polymer includes the nutrient phase. This may be achieved by co-blending the nutrient phase with the fluoropolymer resin prior to expansion of the polymer.

In some embodiments, the cultivation substrate comprises a polymer formed by expanded chemical vapor deposition (CVD). In some embodiments, the polymer formed by expanded CVD forms the microstructure of the cultivation substrate. In some embodiments, the polymer formed by expanded CVD is polyparaxylylene (ePPX).

In some embodiments, the cultivation systems described herein can be used to germinate spores. Spores are contacted for a sufficient time and under predetermined conditions with a cultivation substrate having desired properties for retaining and viably maintaining the spores until at least some of the spores are retained within the microstructure of the cultivation substrate. In some embodiments, upon retention of the spores by the cultivation substrate, the cultivation substrate can be incubated in a medium conducive to the germination of the spores and growth of the germinated spores. In other embodiments, the culture system itself provides a microenvironment conducive to the germination of spores and growth of the germinated spores, at least for a period of time (e.g., during temporary transport).

In some embodiments, the cultivation substrates described herein can be used as a growth substrate for multicellular organisms from spores. For example, the cultivation substrates can be used to support growth of seaweed from spore to mature seaweed. In some embodiments, the spore that is to mature into the multicellular organism is contacted for a sufficient time and under predetermined conditions with a cultivation substrate having desired properties for retaining and viably maintaining the spores and supporting growth of a multicellular organism therefrom, until at least some of the spores are retained within the microstructure of the cultivation substrate.

In some embodiments, seaweed spores are introduced into the microstructure of the cultivation substrate, and gametophytes and sporophytes are allowed to mature in a manner similar to traditional culture strings, where spores are introduced to the cultivation substrate in a laboratory setting. Alternatively, the spores are introduced to the microstructure of the cultivation substrate in the field (i.e., at the seaweed farm site). This is achieved due to the retention properties of the microstructure of the cultivation substrate. By depositing a material having the presently described microstructure (either with or without spores retained therein) on a rope, cable, or other support in the field, the traditional step of wrapping a culture string around a rope line can be skipped. This can be accomplished where the microstructure is provided by a plurality of particles in a dispersion.

In other embodiments, seaweed sporophytes and/or gametophytes are directly introduced into the microstructure of the cultivation substrate. Such direct seeding can reduce the laboratory time required to produce a culture string relative to spore seeding.

Culture strings are traditionally maintained and cultured in a laboratory environment using sterilized sea water. The present cultivation systems, through inclusion of sufficient salt within the microstructure, circumvents the need for the expensive and cumbersome systems required for circulation of sterilized sea water by providing a saline microenvironment within the microstructure. In some embodiments, the cultivation substrate and retained spores are maintained in a standard seaweed cultivation tank, where nutrients are delivered via sterile seawater. By including a nutrient phase within the microstructure sufficient to support seaweed growth, the need to provide external nutrients to the growing seaweed may be obviated.

Culture strings must be carefully transported in sea water while avoiding jostling to prevent gametophyte and sporophyte detachment from the string. Conversely, the presently described cultivation systems allow for the gametophytes and sporophytes to be safely transported without sea water. This is achievable by the inclusion of salt and a liquid containing phase within the microstructure, which provides a saline microenvironment having sufficient moisture to support the juvenile seaweed during transport. Furthermore, as the juvenile seaweed is able to grow into the microstructure rather than simply attach superficially to a surface of, e.g., a culture string, loss by detachment is minimized. This beneficial effect extends to the seaweed farm, where currents may detach weakly secured juvenile seaweed.

### Examples

### Example 1—Porous Polyethylene

Dulse and kelp cultivation trials were conducted on 2 porous polyethylene-based membranes.

Membrane 1 is a gel processed polyethylene membrane measuring 500 millimeters wide, 30 microns thick, with an area density of 18.1 g/m² and an approximate porosity of 36%. This tape was subsequently stretched in the machine direction through a hot air dryer set to 120 degrees Celsius at a stretch ratio of 2:1 with a stretch rate of 4.3%/second. This was followed by a transverse direction stretch in an oven at 130 degrees Celsius at a ratio of 4.7:1 with a stretch rate of 15.6%/second. The resulting membrane possessed the following properties: width of 697 millimeters, thickness of 14 microns, porosity of 66%, and maximum load of 7.65 Newtons×6.23 Newtons and elongation at maximum load of 25.6%×34.3% in the machine direction and transverse directions respectively as tested according to ASTM D412. The membrane had a Gurley Time of 15.7 seconds. Gurley Time is defined as the number of seconds required for 100 cubic centimeters (1 deciliter) of air to pass through 1.0 square inch of a given material at a pressure differential of 4.88 inches of water (0.176 psi) (ISO 5636-5:2003).

Membrane 2 is a commercially available porous polyethylene from Saint Gobain rated as a UE 1 micron lab filter disc. The microstructure of membrane 2 is depicted in FIG. 13.

Membrane samples were secured to 2 inch diameter PVC cups. All samples were sprayed with alcohol and rinsed with freshwater just prior to seeding. Seeding was accomplished by pouring spore solution over samples and allowing spores to settle onto substrate surfaces. Samples were seeded in 10 gallon tanks, and seawater was changed every week. Dulse samples were moved to a 40 gallon fiberglass tank after week 2. Kelp were cultured in 10 gallon tanks. All cultures received aeration. Samples were photographed 2 months after seeding when plants were visible.

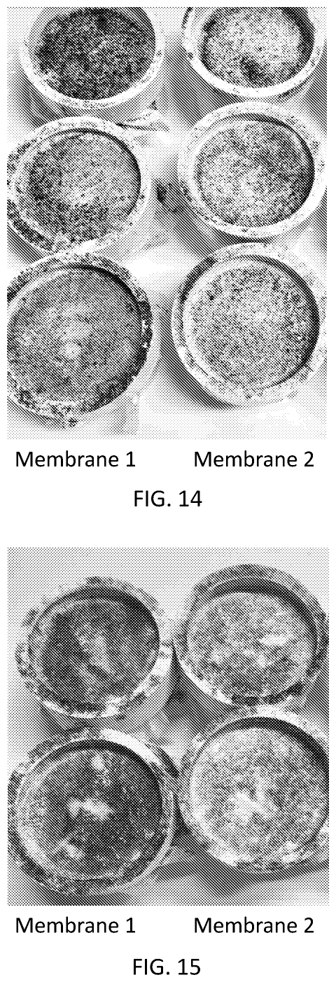

All dulse samples were gently rinsed with freshwater and then dipped into seawater before the evaluation to remove any fouling. Both membrane 1 and 2 showed healthy, medium to high density growth of dulse seedlings (see FIG. 14). Membrane 1 showed higher density plant growth than Membrane 2. Both Membrane 1 and 2 showed strong seedling attachment and stability.

Kelp samples were lightly rinsed with seawater before photographing. Both membrane 1 and 2 showed healthy, medium to high density growth of Kelp seedlings (see FIG. 15). Membrane 1 showed higher density plant growth than Membrane 2. Both Membrane 1 and 2 showed strong seedling attachment and stability.

### Example 2—Patterned Membranes

A patterned fluoropolymer-based membrane in accordance with certain embodiments was generated with large square areas of low and high porosity. The pattern was in the form of a “checkerboard” design.

Membrane samples were secured to 2 inch diameter PVC cups. All samples were sprayed with alcohol and rinsed with freshwater just prior to seeding. Seeding was accomplished by pouring spore solution over samples and allowing spores to settle onto substrate surfaces. Samples were seeded in 10 gallon tanks, and seawater was changed every week. Dulse samples were moved to a 40 gallon fiberglass tank after week 2. Kelp samples were cultured in 10 gallon tanks. All cultures received aeration. Samples were photographed 2 months after seeding when plants were visible.

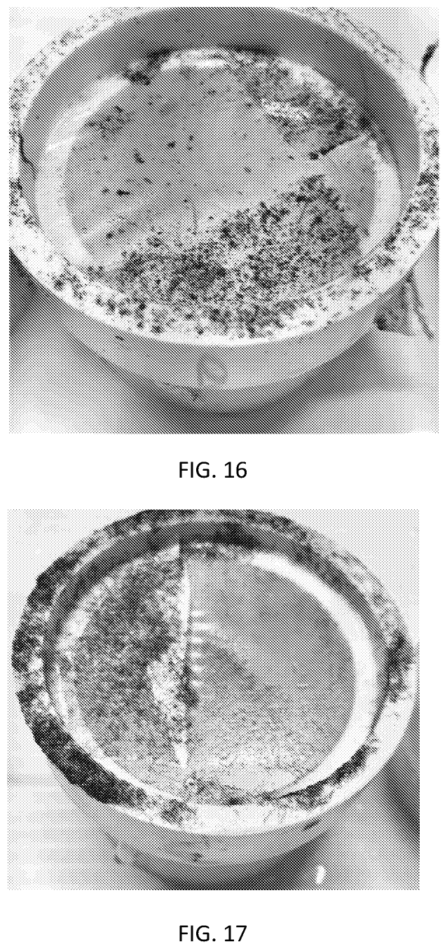

All dulse samples were gently rinsed with freshwater and then dipped into seawater before the evaluation to remove any fouling. With reference to FIG. 16, the checkerboard pattern showed large differences in plant density, with the high porosity (white) squares supporting a healthy, high density covering of plants with strong attachment and the low porosity (clear) squares showing a very low density covering of plants.

Kelp samples were lightly rinsed with seawater before photographing. With reference to FIG. 17, the checkerboard pattern showed large differences in plant density, with the high porosity (white) squares supporting a healthy, high density covering of plants with strong attachment and the low porosity (clear) squares showing a very low density covering of plants.

### Example 3—Direct Sporophyte Seeding

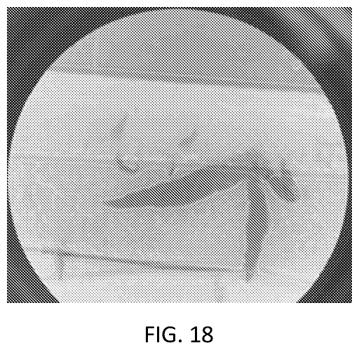

Juvenile sugar kelp sporophytes previously in induction conditions were seeded without any binder onto an experimental membrane of the present disclosure having a width of 4 mm, and a braided polyester control having a diameter of 2 mm. Attachment of the juvenile sporophytes was evaluated for 19 days after seeding. The sporophytes demonstrated attachment and growth on both substrates. Healthy sporophyte growth on the membrane of the present disclosure is depicted in FIG. 18.

To quantify the attachment strength to the two substrates, scores on a scale of 1 to 5 were given to 20 or more sporophytes attached to each substrate, with 1 being very weak attachment and 5 being very strong attachment. The majority of sporophytes attached to the braided polyester control were rated ‘1’, with very weak attachment. The majority of sporophytes attached to the experimental membrane were rated ‘5’, with very strong attachment. The difference in attachment strength between the two substrates was further demonstrated by the ability of sporophytes attached to the experimental membrane to be handed and moved with tweezers while remaining attached to the substrate. The sporophytes attached to the braided polyester control could not be handled, moved, or even agitated without being detached from the substrate.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A cultivation system comprising a cultivation substrate having a microstructure configured to retain and viably maintain spores, the microstructure being characterized by an average inter-fibril distance up to and including 200 μm.

2. A cultivation system comprising a cultivation substrate having a microstructure wherein at least a portion of the cultivation system is configured to retain and viably maintain, the microstructure configured to retain spores at least partially within the microstructure of the cultivation substrate, the microstructure being characterized by an average pore size of up to and including 200 μm.

3. The cultivation system of claim 1, wherein the microstructure is characterized by an average inter-fibril distance from 1 to 200 μm.

4. The cultivation system of claim 1 or claim 2, wherein the microstructure is characterized by an average pore size from 1 to 200 μm.

5. The cultivation system of any one of claims 1-4, further comprising a nutrient phase associated with at least a portion of the cultivation substrate.

6. The cultivation system of claim 5, wherein at least a portion of the nutrient phase is located within the cultivation substrate, located on the cultivation substrate, or located within the cultivation substrate and on the cultivation substrate.

7. The cultivation system of claim 5, wherein the nutrient phase is present as a coating on a surface of the cultivation substrate.

8. The cultivation system of any one of claims 5-7, wherein the nutrient phase acts as a chemoattractant to selectively attract the spores to predetermined locations of the cultivation substrate to which the nutrient phase is applied or included.

9. The cultivation system of any of claims 5-8, wherein the nutrient phase is configured to i) promote germination of and growth from the spores within the microstructure, and/or ii) maintain and/or encourage attachment to and integration within the microstructure by the spores.

10. The cultivation system of any one of claims 1-9, further comprising a liquid containing phase associated with at least a portion of the cultivation substrate.

11. The cultivation system of claim 10, wherein at least a portion of the liquid containing phase is entrained within the microstructure, entrained on the microstructure, or entrained within the microstructure and on the microstructure.

12. The cultivation system of claim 10 or claim 11, wherein the liquid containing phase is present as a coating on a surface of the cultivation substrate.

13. The cultivation system of any one of claims 10-12, wherein the liquid containing phase comprises a hydrogel, a slurry, a paste, or a combination thereof.

14. The cultivation system of any one of claims 1-13, further comprising a plurality of spores, germinated spores, or both spores and germinated retained by the microstructure of the cultivation substrate.

15. The cultivation system of any one of claims 1-14, wherein the cultivation substrate includes a fibrillated material having a microstructure including a plurality of fibrils defining an average inter-fibril distance.

16. The cultivation system of any one of claims 1-15, wherein the microstructure of the cultivation substrate is configured to retain spores having an average spore size of up to and including 200 μm.

17. The cultivation system of any one of claims 1-16, wherein the spores comprise algal spores.

18. The cultivation system of any one of claims 1-16, wherein the spores comprise fungal spores.

19. The cultivation system of any one of claims 1-16, wherein the spores comprise plant spores.

20. The cultivation system of any one of claims 1-19, wherein the cultivation substrate comprises a material having an average density from 0.1 to 1.0 g/cm³.

21. The cultivation system of claim 20, wherein the cultivation substrate includes a growth medium comprising the material, and a ratio of the average inter-fibril distance (μm) to the average density (g/cm³) of the fibrillated material is from 1 to 2000.

22. The cultivation system of any one of claims 1-21, wherein the cultivation substrate is configured as a fiber, a membrane, a woven article, a non-woven article, a braided article, a knit article, a fabric, a particulate dispersion, or combinations of two or more of the foregoing.

23. The cultivation system of any one of claims 1-22, wherein the cultivation substrate includes at least one of a backer layer, a carrier layer, a laminate of a plurality of layers, a composite material, or combinations thereof.

24. The cultivation system of any one of claims 1-23, wherein at least a portion of the cultivation substrate is hydrophilic.

25. The cultivation system of any one of claims 1-24, wherein at least a portion of the cultivation substrate is hydrophobic.

26. The cultivation system of any one of claims 1-25, wherein one or more portions of the cultivation substrate is hydrophobic and one or more portions of the cultivation system is hydrophilic such that the cultivation system is configured to selectively encourage spore retention in the one or more hydrophilic portions of the cultivation substrate.

27. The cultivation system of any one of claims 1-26, wherein the cultivation substrate comprises an expanded fluoropolymer.

28. The cultivation system of any one of claims 1-27, wherein the expanded fluoropolymer is one of: expanded fluorinated ethylene propylene (eFEP), porous perfluoroalkoxy alkane (PFA), expanded ethylene tetrafluoroethylene (eETFE), expanded vinylidene fluoride co-tetrafluoroethylene or trifluoroethylene polymer (eVDF-co-(TFE or TrFE)), and expanded polytetrafluoroethylene (ePTFE).

29. The cultivation system of any one of claims 1-26, wherein the cultivation substrate comprises an expanded thermoplastic polymer.

30. The cultivation system of claim 29, wherein the expanded thermoplastic polymer is one of: expanded polyester sulfone (ePES), expanded ultra-high-molecular-weight polyethylene (eUHMWPE), expanded polylactic acid (ePLA), and expanded polyethylene (ePE).

31. The cultivation system of any one of claims 1-26, wherein the cultivation substrate comprises an expanded polymer.

32. The cultivation system of claim 31, wherein the expanded polymer is expanded polyurethane (ePU).

33. The cultivation system of any one of claims 1-26, wherein the cultivation substrate comprises a polymer formed by expanded chemical vapor deposition (CVD).

34. The cultivation system of claim 33, wherein the cultivation substrate is expanded polyparaxylylene (ePPX).

35. The cultivation system of any one of claims 1-34, further comprising a bioactive agent associated with the cultivation substrate.

36. The cultivation system of any one of claims 1-35, further comprising an adhesive applied to a surface of the cultivation substrate, imbibed within the microstructure of the cultivation substrate, or both applied to a surface of the cultivation substrate and imbibed within the microstructure of the cultivation substrate.

37. The cultivation system of any one of claims 1-37, further comprising a salt associated with the cultivation substrate.

38. The cultivation system of claim 37, wherein the salt is sodium chloride (NaCl).

39. The cultivation system of any one of claims 1-38, wherein the cultivation substrate includes a pattern of higher density portions and lower density portions, the lower density portions corresponding to a portion of the cultivation substrate configured to retain spores at least partially within the microstructure of the cultivation substrate.

40. The cultivation system of claim 39, wherein the lower density areas are characterized by a density of 1 g/cm³ or less and the higher density portions are characterized by a density of 1.7 g/cm³ or more.

41. The cultivation system of any one of claims 1-40, wherein the cultivation substrate includes a pattern of higher porosity portions and lower porosity portions, the lower porosity portions corresponding to a portion of the cultivation substrate configured to retain spores within the microstructure of the cultivation substrate.

42. The cultivation system of any one of claims 1-40, wherein the cultivation substrate includes a pattern of higher porosity portions and lower porosity portions, the higher porosity portions corresponding to a portion of the cultivation substrate configured to retain spores within the microstructure of the cultivation substrate.

43. The cultivation system of any one of claims 1-42, wherein the cultivation substrate includes a pattern of greater inter-fibril distance portions and lower inter-fibril distance portions, the lower inter-fibril distance portions corresponding to the portion of the cultivation substrate configured to retain spores within the microstructure of the cultivation substrate.

44. The cultivation system of any one of claims 1-42, wherein the cultivation substrate includes a pattern of greater inter-fibril distance portions and lower inter-fibril distance portions, the greater inter-fibril distance portions corresponding to the portion of the cultivation substrate configured to retain spores within the microstructure of the cultivation substrate.

45. The cultivation system of claim 43 or claim 44, wherein the pattern is an organized or selective pattern.

46. The cultivation system of claim 43 or claim 44, wherein the pattern is a random pattern.

47. The cultivation system of any one of claims 1-46, wherein the microstructure is initially in a first retention phase to retain the spores and subsequently in a second growth phase to induce ingrowth of sporelings from the spores on and/or into the microstructure to mechanically couple the sporelings to the microstructure.

48. The cultivation system of any one of claims 1-47, wherein nutrients are configured to be delivered via sterile seawater.

49. The cultivation system of any one of claims 1-48, wherein the microstructure is configured to irremovably anchor a portion of each of the spores.

50. The cultivation system of any one of claims 1-49 wherein the microstructure is configured to irremovably anchor germinated spores.

51. The cultivation system of any one of claims 1-50, wherein the cultivation substrate is provided by a plurality of particles in a dispersion formulated for deposition onto a backer layer or carrier substrate.

52. A method for cultivating seaweed, comprising contacting a population of seaweed spores, gametophytes, or sporophytes with the cultivation system of any one of claims 1-51 until at least a portion of the population of seaweed spores, gametophytes, or sporophytes is retained by the cultivation system.

53. The method of claim 52, further comprising positioning the cultivation system including a portion of the population of seaweed spores, gametophytes, or sporophytes in an open-water environment.
